(19) United States
(12) Patent Application Publication
(10) Pub. No.: US 2022/0202853 A1
(43) Pub. Date: Jun. 30, 2022

(54) HYPOHALOUS ACIDS FOR TREATING INFLAMMATORY DISEASES AND INHIBITING GROWTH OF MALIGNANCIES

(71) Applicant: Briotech, Inc., Woodinville, WA (US)

(72) Inventors: Richard Jeremy Stone, London (GB); Lori Robins, Sammamish, WA (US); Jeffrey Francis Williams, Langley, WA (US); Daniel James Terry, Mill Creek, WA (US); Eric Rasmussen, Kirkland, WA (US)

(21) Appl. No.: 17/562,871

(22) Filed: Dec. 27, 2021

Related U.S. Application Data
(60) Provisional application No. 63/131,210, filed on Dec. 28, 2020.

(51) Int. Cl. A61K 33/20; A61K 33/22

(52) U.S. Cl. CPC A61K 33/20; A61K 33/22

(57) ABSTRACT

A method of neutralizing adverse biological effects of Interleukin 6 (IL-6) and other pro-inflammatory Interleukins in vivo is disclosed using homogeneous solutions of hypohalous acids to modify binding sites on cytokines and chemokines that are responsible for activation of cell surface receptors, and the initiation of harmful inflammatory processes or the growth of cytokine-dependent malignancies or of malignancies dependent on other chemical growth factors.

### TECHNICAL FIELD

The present disclosure generally relates to hypohalous acid compositions and their use for treatment and prevention of inflammatory diseases caused by the in vivo biological activities of the mammalian cytokine Interleukin 6 (IL-6) and other chemical factors involved in the development of inflammatory and neoplastic lesions.

### BACKGROUND

Interleukin 6 (IL-6) is a 26 kDa pleiotropic pro-inflammatory cytokine produced by a variety of mammalian cell types, including fibroblasts, monocytes and endothelial cells. These are the main source of IL-6 in vivo. In addition, cells such as T cells, B cells, macrophages, keratinocytes, osteoblasts and several others can produce IL-6 when suitably stimulated.

Cell signaling in response to IL-6 is initiated by binding of IL-6 to a transmembrane receptor, IL-6 receptor alpha (also referred to as IL-6Ra, IL-6Ra, IL-6R, gp80 or CD126). A complex is formed termed “IL-6:IL-6Ra”. This complex binds to the gp130 signal receptor; IL-6Rα and gp130 together form a high affinity IL-6 binding site. IL-6Ra can also exist as a soluble secreted entity (sIL-6R), which can further associate with IL-6 to form a ligand-receptor complex. This complex can bind to gp130, and by this means activate a wide array of cell types, accelerating and broadening inflammatory responsiveness to injury and infection.

The crystal structure of human IL-6 has been determined. There are three sites on the surface of IL-6 involved in the functional activity of the IL-6 in association with receptor components. Site 1 residues are involved in the interaction between IL-6 and IL-6Ra. Site 2 residues are involved in the interaction between IL-6 and the gp130 cytokine binding domain. The residues in Site 3 of IL-6 are involved in interacting with the Ig-like domain of the second gp130 in a hexameric complex.

IL-6 shows a wide spectrum of biological functions including: hematopoiesis, induction of acute phase responses, T cell activation, stimulation of antibody secretion, host defense against infection, myeloma cell and osteoclast activation. Although IL-6 was originally identified as a B-cell differentiation factor generated by T cells it has subsequently been identified as a potent activator and growth-promoting factor of many other cell types. It induces maturation of B lymphocytes, and is an accessory factor for T cell activation and proliferation. This cytokine is involved in the activation of auto-reactive T lymphocytes and the proliferation and differentiation of cytotoxic T cells. IL-6 induces a variety of acute phase proteins such as fibrinogen, alpha-anti-chymotrypsin, serum amyloid A and C-reactive protein, all of which can be involved in immune responses and inflammation.

Because IL-6 has such a variety of biological effects, elevation of this cytokine has been implicated as causal in a variety of disease processes, including most recently in the pathogenesis of acute, severe COVID-19 pulmonary lesions that often prove fatal. There is evidence of its involvement in rheumatoid arthritis, polymyalgia rheumatica, fibromyalgia, myalgic encephalomyelitis, demyelinating conditions such as multiple sclerosis and optic neuritis, acute respiratory distress syndrome (ARDS), disseminated intravascular coagulation, and the persistent clinical conditions associated with post COVID-19 disease.

Concentrations of circulating IL-6 are markedly elevated in rheumatoid arthritis (RA), Castleman's disease, juvenile idiopathic arthritis and Crohn's disease. Certain malignancies are also known to be responsive to IL-6, and increased concentrations of IL-6 are seen in the plasma of some cancer patients. These include prostate cancer, multiple myelomas, plasma cell leukemias and several types of carcinomas.

In view of this array of likely involvements of IL-6 in pathogenetic mechanisms, it is not surprising that many efforts have been made in recent years to develop and apply antagonists that can intervene in the IL-6/receptor interactions. These have been aimed at modifying or blocking either the cytokine in its capacity to bind to cell receptors or the receptor components that can lead to activation of inflammatory cell participants or promote malignant growths. The goal has been to treat or prevent events in vivo that result from IL-6-dependent disease mechanisms. The great majority of these efforts have been focused on using antibodies or antibody fragments specifically targeting epitopes on IL-6 or on components of the receptor complex. Appropriately specific immune reagents are then administered systemically to bring about inhibition at disease sites wherever they are in process.

Although molecular biological technology has allowed for the development and introduction of antibody-based therapeutic products with proven effects on many of the conditions identified above, these pharmaceutical reagents are expensive to manufacture, complicated to distribute and deliver, and prone to generate significant adverse reactions in an important proportion of recipients. These characteristics make the wide scale adoption of antibody-based approaches to IL-6-induced pathologies problematic, particularly in regions of the world where healthcare practices cannot accommodate costly products or cope readily with severe side effects of their use. Alternative approaches to mitigation of the potential for damage caused by this important cytokine are needed.

Few studies have approached possible antagonistic effects of reactants directly on IL-6 or its corresponding receptor, or the potential for topical intervention in ongoing pathological processes dependent on these participants. Hypochlorous acid (HOCl) is known to react with proteins and polypeptides by way of oxidation or chlorination of amino acid residues, and such modifications can result in a wide range of effects on the state and functionality of these macromolecules. Proteins such as human serum albumin, for example, may acquire new affinities for viral surfaces after exposure to HOCl, and the immunogenicity of staphylococcal antigens is increased once chlorination of certain constituent amino acids occurs upon HOCl treatment.

Hypohalous acids, such as HOCl and hypobromous acid (HOBr), are natural products of the innate immune system of mammals and many other animals, generated on demand as a result of intracellular myeloperoxidase activation in phagosomes of granulocytes, macrophages and microglial cells. These end products of the so-called respiratory burst that accompanies infection or injury to tissues exert powerful, broad-spectrum antimicrobial properties, before they are consumed by intra and extracellular substrates. The reaction products of those substrates trigger the healing and restoration processes that follow on injury to tissues.

Hypochlorous acid can be produced commercially and was recognized for its role in these protective events years ago. However, the practical utility of hypochlorous acid has been severely limited by an historical reputation for instability and its tendency to degrade rapidly into ineffective and cytotoxic reaction products. This traditional severe instability of hypochlorous acid and tenancy to rapidly degrade into ineffective and cytotoxic reaction products has made hypochlorous acid not useful and even potential hazardous for many applications due to the cytotoxic reaction products that can be produced from the rapid degrading of the hypochlorous acid. There is a continuing need for hypochlorous acid without these

### BRIEF SUMMARY

Briefly stated, the presently disclosed embodiments have overcome the limitation of hypochlorous acid having serious instability and its tendency to degrade rapidly into ineffective and cytotoxic reaction products, by using a novel electrochemistry technique. Such presently disclosed homogeneous preparations of HOCl at pH 4 are stable, potent, and safe for topical use on skin and wounds, and can be inhaled as aerosols so as to deliver HOCl to both upper and lower respiratory system epithelial surfaces. HOCl is readily converted to HOBr by addition of equimolar amounts of sodium or potassium bromide. While HOBr is equally or more effective as an antimicrobial agent than HOCl, and provides comparable benefits when applied topically, it is very short-lived and unstable, deteriorating in hours to form ill-defined mixtures of aqueous bromine species. Both HOCl and HOBr react quickly but differently with amino acid residues that may form part of the receptor binding domains (RBD) of proteins and polypeptides, and are therefore candidates for modification or inactivation of biological activities associated with these kinds of structures, including cytokines.

The present disclosure is directed to compositions comprising authentic pure hypohalous acids (e.g., not contaminated with hypochlorites or hypobromites) capable of rapidly inactivating IL-6, the key cytokine involved in the pathogenesis of many disease states, including the acute pulmonary events that lead to overwhelming lung damage in COVID-19 infections. Inactivation of IL-6 by hypohalous acids applied topically or through systemic delivery, e.g., via inhalation of aerosols of hypohalous acids, can also be used to aid in the control of certain malignancies that are dependent on this cytokine for stimulation of growth and replication of cancerous cells. Since other Interleukins and chemokines participate in pro-inflammatory events, cytokine storms (e.g., IL-2, IL-7), and in some cases act as growth factors for cancer cell types (e.g., IL-2) the likelihood is high that they will be similarly affected by the compositions and use conditions described herein.

Accordingly, one embodiment of the present disclosure is directed to a method for preventing, alleviating, reducing or treating an inflammatory disorder, the method comprising administering to a subject an effective amount of a composition comprising a hypohalous acid, wherein the composition is substantially free of hypochlorite and hypobromite.

In some embodiments, the inflammatory disorder is derived from rheumatoid arthritis, polymyalgia rheumatica, fibromyalgia, myalgic encephalomyeltis, multiple sclerosis, optic neuritis, acute respiratory distress syndrome (ARDS), disseminated intravascular coagulation, acute smoke inhalation, COVID-19 or combinations thereof.

Another embodiment of the present disclosure is directed to a method for inhibiting or reducing the growth of a cancer, the method comprising administering to a subject an effective amount of a composition comprising a hypohalous acid and a pharmaceutically acceptable carrier, wherein the composition is substantially free of hypochlorite and hypobromite.

In some embodiments, the cancer is selected from the group consisting of prostate cancer, multiple myeloma, plasma cell leukemia, carcinomata, and sarcomata.

In one or more embodiments, the composition inhibits biological activities of Interleukin 6 (IL-6), other Interleukins, and chemokines in vivo.

In some embodiments, the composition is an aqueous solution having a hypohalous acid concentration from about 5 mg/L to about 5000 mg/L. In other embodiments, the composition is an aqueous solution having a pH from about 3.8 to about 6.5. In still other embodiments, the composition has a pH from about 4.0 to about 4.2. In yet other embodiments, the composition has a pH of about 4.0.

In some embodiments, the composition is an aqueous solution having an oxidative reduction potential (ORP) from about +900 milivolts to +1200 milivolts. In other embodiments, the composition does not contain a detectable amount of hypochlorite and hypobromite. In still other embodiments, an amount of hypochlorite and hypobromite in the composition is less than 200 ppm.

In some embodiments, the hypohalous acid is hypochlorous acid. In other embodiments, the composition has a hypochlorous acid concentration from about 5 mg/L to about 500 mg/L, a pH from about 3.8 to about 6.5 and an ORP of about +1000 millivolts. In still other embodiments, the composition has a hypochlorous acid concentration from about 80 mg/L to about 300 mg/L, a pH from about 4.0 to about 4.2, and an ORP of about +1100 millivolts. In yet other embodiments, the composition has a hypochlorous acid concentration from about 80 mg/L to about 300 mg/L, a pH from about 4.0 to about 4.2, and an ORP of about +1138 mV.

In some embodiments, the hypohalous acid is hypobromous acid. In other embodiments, the composition has a hypobromous acid concentration from about 5 mg/L to about 300 mg/L, a pH from about 4.0 to about 7.5, an oxidative reduction potential (ORP) of about +1000 millivolts. In still other embodiments, the composition has a hypobromous acid concentration from about 5 mg/L to about 350 mg/L, a pH from about 4.0 to about 7.5, an oxidative reduction potential (ORP) of about +900 millivolts.

In some embodiments, the composition is formulated for topical administration. In other embodiments, the composition is formulated for administration via inhalation. In still other embodiments, the composition is formulated as a liquid, a gel, a lotion, a cream, a foam, or an aerosol spray. In yet other embodiments, the composition further comprises a pharmaceutically acceptable carrier. In other embodiments, the carrier comprises a clay, a cellulose, a silicate or a combination thereof.

In some embodiments, the composition is administered from 1 to 10 times per day. In other embodiments, the composition is administered from 1 to 4 times per day. In still other embodiments, the composition is an aqueous hypochlorous acid composition prepared by electrolysis of an aqueous solution of sodium or potassium chloride. In yet other embodiments, the composition further comprises converting the hypochlorous acid to hypobromous acid prior to the administration of the composition to the subject. In some embodiments, converting the hypochlorous acid to the hypobromous acid comprises mixing the hypochlorous acid composition with an aqueous solution of sodium or potassium bromide in equimolar amounts.

### BRIEF DESCRIPTION OF THE SEVERAL VIEWS OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 illustrates inhibition of IL-6 binding by exposure to HOCl.

FIG. 2 illustrates inhibition of IL-6 binding by exposure to HOBr.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### Definitions

As used herein, and unless the context dictates otherwise, the following terms have the meanings as specified below.

“Preventing”, “prevention” and “prevent” in the context of the disclosed methods all refer to prophylactic methods which hinder or stop the occurrence of a particular condition, for example inflammatory disorder or cancer.

“Alleviating”, “alleviation” and “alleviate” in the context of the disclosed methods all refer to lessening or mitigating the effects or symptoms of a particular condition, for example inflammatory disorder or cancer.

“Reducing”, “reduction” and “reduce” in the context of the disclosed methods all refer to decreasing the effects or symptoms of a particular condition, for example inflammatory disorder or cancer.

“Treating”, “treatment” and “treat” in the context of the disclosed methods all refer to techniques or methods intended to improve the symptoms of or decrease or stop the occurrence of a particular condition, for example inflammatory disorder or cancer.

An “Effective amount” in the contact of the disclosed methods refers to an amount of hypohalous acid which is effective for treating, alleviating, ameliorating, relieving, delaying onset of, inhibiting progression of, reducing severity of, and/or reducing incidence of one or more symptoms or features of a disease, for example inflammatory disorder or cancer.

“Inhibition of growth” can also refer to a reduction in size or disappearance of a cancer cell or tumor, as well as to a reduction in its metastatic potential. Preferably, such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

A “host” or “subject” or “patient” is a living subject, human or animal, into which the compositions described herein are administered. Thus, the compositions described herein may be used for veterinary as well as human applications and the terms “patient” or “subject” or “host” should not be construed in a limiting manner.

In some embodiments, the term “about” means with 10% of the target value. In other embodiments, the term “about” means with 5% of the target value. In still other embodiments, the term “about” means with 1% of the target value. In yet other embodiments, the term “about” means with 0.1% of the target value.

As noted above, one embodiment of the present disclosure relates to use of a composition comprising an authentic pure and stable hypohalous acid for prevention, alleviation, reduction or treatment of an inflammatory disorder.

Another embodiment of the present disclosure relates to use of a composition comprising an authentic pure and stable hypohalous acid for prevention, alleviation, reduction or treatment of a cancer. These and other aspects and various embodiments of the present disclosure will become evident upon reference to the description which follows.

In all cases, inactivation is effected by oxidation and or halogenation of key sites on IL-6 and other Interleukins and chemokines by hypohalous acid that alter the functionality of receptor binding domains that are responsible for triggering cell responsiveness to the cytokine via interaction with membrane bound receptors. In this way pathogenetic pathways responsible for a wide array of disease manifestations can be inhibited or prevented altogether, enabling novel therapeutic or prophylactic interventions in ways that do not depend on conventional immunological reagents that are expensive to make and deliver, and provoke significant side effects.

### A. Hypohalous Acid Composition

The methods of the present disclosure utilize a hypohalous acid composition. As defined herein, the authentic pure hypohalous acid means the hypohalous acid composition is substantially free of hypochlorite and hypobromite, as well as substantially free of pH buffers. That is, the hypohalous acid composition does not contain a detectable amount of hypochlorite and hypobromite and is produced so as to exclude the use of buffers, metal ions, organic heterocyclic halogen stabilizers or pH modifiers of any sort, at any level.

In some embodiments, the composition comprises hypohalous acid at a concentration from about 5 mg/L to about 500 mg/L, from about 10 mg/L to about 450 mg/L, from about 50 mg/L to about 400 mg/L, from about 80 mg/L to about 300 mg/L, from about 100 mg/L to about 200 mg/L, or from about 120 mg/L to about 180 mg/L.

In some embodiments, the composition has a pH from about 3.8 to about 6.5, from about 4.0 to about 5, from about 4.0 to about 4.2. In some embodiments, the composition has a pH of about 4.

In some embodiments, the composition has an oxidative reduction potential (ORP) from about +900 millivolts (mV) to about 1200 mV. In some embodiments, the composition has an ORP of about +1000 mV. In other embodiments, the composition has an ORP of about +1100 mV. In still other embodiments, the composition has an ORP of about 1138 mV.

In some embodiments, the hypohalous acid is hypochlorous acid. In certain of these embodiments, the composition comprises hypochlorous acid at a concentration from about 5 mg/L to about 500 mg/L, and has a pH from about 3.8 to about 6.5, and an ORP of about +1000 millivolts. In other of these embodiments, the composition comprises hypochlorous acid at a concentration from about 80 mg/L to about 300 mg/L, and has a pH from about 3.8 to about 5.0, and an oxidative reduction potential (ORP) of about +1100 millivolts. In further of these embodiments, the composition comprises hypochlorous acid at a concentration from about 80 mg/L to about 300 mg/L, and has a pH from about 4.0 to about 4.3, and an oxidative reduction potential (ORP) of about +1138 millivolts.

In some embodiments, the hypohalous acid is hypobromous acid. In certain of these embodiments, the composition comprises hypobromous acid at a concentration from about 10 mg/L to about 300 mg/L, and has a pH from about 4.0 to about 7.5, and an oxidative reduction potential (ORP) of about +1000 millivolts. In other of these embodiments, the composition comprises hypobromous acid at a concentration from about 5 mg/L to about 350 mg/L, and has a pH from about 4.0 to about 7.5, and an oxidative reduction potential (ORP) of about +900 millivolts.

The hypohalous acid composition does not contain a detectable amount of hypochlorite and hypobromite as measured by Raman spectroscopy at 715-740 centimeters⁻¹, preferably at 715-732 centimeters⁻¹ and at 615-640 centimeters⁻¹. A singular 720-740 or 615-640 centimeters⁻¹ Raman signal indicates the presence of only hypochlorous acid or only hypobromous acid (i.e., no hypochlorite or hypobromite) having a pH from about 4.0 to 7.5 and a state of isotonicity, respectively. In some embodiments, the composition includes less than 500 ppm, less than 400 ppm, less than 300 ppm, less than 200 ppm, less than 100 ppm, or less than 50 ppm hypochlorite and hypobromite. In certain embodiments, the composition includes less than 100 ppm hypochlorite and hypobromite. In other embodiments, the composition includes less than 100 ppm hypochlorite and hypobromite. In further embodiments, the composition includes less than 50 ppm hypochlorite and hypobromite. In yet additional embodiments, the composition is free from detectable amount of hypochlorites and hypobromites, as determined by Raman spectroscopy.

The absence of detectable amounts of hypochlorite and hypobromite contributes to stability of the hypochlorous acid composition by the avoidance of acceleration of reactions that degrade hypochlorous acid or hypobromous acid. Such stability relates to the primary values in hypohalous acid shelf stability in terms of the concentration of hypohalous acid in parts per million, ORP, pH and thermal tolerance from −80° C. to 100° C.

In some embodiments, the hypohalous acid is hypochlorous acid and is stable at room temperature, freezing temperatures (i.e., −80° C.) and high temperatures (i.e., 80° C.). As defined herein, stable means that the hypochlorous acid composition described herein within a sealed container, has a detectable loss of ORP after 36 months of storage at 25° C. that is less than 10%, preferably less than 5%, and more preferably 0%. Additionally, as defined herein, stable means that the hypochlorous acid composition described herein within a sealed impervious container, has a detectable loss of hypochlorous acid after 36 months of storage at 25° C. that is less than 50% and still more preferably less than 25%. Furthermore, as defined herein, stable means that the HOCl composition described herein within a sealed impervious container, has no measureable hypochlorous or oxidants after 36 months of storage at 25° C.

In certain embodiments, the hypohalous acid is hypochlorous acid and the composition has a shelf life of useful inactivation efficacy up to about 36 months in a sealed impervious container. In other embodiments, the hypohalous acid is a predominantly hypobromous acid and the composition has a shelf life of useful inactivation efficacy of about four to about six hours in a sealed impervious container.

In some embodiments, the hypohalous composition does not contain any additives such as buffer or hypohalous acid stabilizer. For example, in some embodiments, the hypohalous acid composition does not include a mono- or di-phosphate sodium or potassium buffer, a carbonate buffer, periodate, divalent metal cation, organic heterocyclic compound, hydrochloric acid, hydrobromic acid, or a chemical entity conventionally used as a halogen stabilizer to enhance the stability of a hypohalous acid solution in storage.

The hypochlorous acid may be produced electrochemically. The electrochemical production of hypochlorous acid is carried out by treatment of a chloride-based electrolyte in a hypochlorous acid manufacturing system. Electrochemical production of a chloride-based solution is described, for example, in U.S. Application No. 63/062,287, which is hereby incorporated by reference in its entirety. In some embodiments, the authentic pure hypochlorous acid is produced by using precisely controlled electrolysis of a solution of sodium or potassium chloride (NaCl or KCl). In one or more embodiments, the electrochemical production of hypochlorous acid is carried out using a hypochlorous acid manufacturing system, as described, for example, in U.S. application Ser. No. 17/396,018, which is hereby incorporated by reference in its entirety.

In some embodiments, the authentic pure hypobromous acid is provided by addition of an equimolar amount of sodium or potassium bromide to the hypochlorous acid, thereby converting hypochlorous acid into hypobromous acid. The complete conversion of hypochlorous acid to hypobromous acid can be detected spectrophotometrically by absorption at 250 nm. In some embodiments, the conversion of hypochlorous acid to hypobromous acid is performed at point of use. For example, hypobromous acid is prepared in situ prior to administration to patients.

### B. Formulation of Hypochlorous Acid Compositions

The hypochlorous acid compositions of the present disclosure can be formulated in a variety of ways. In some embodiments, the compositions are formulated for topical administration. In other embodiments, the compositions are formulated for administration via inhalation. In still other embodiments, the compositions are formulated as a liquid, a gel, a lotion, a cream, a foam, or an aerosol or mist spray.

In yet other embodiments, the compositions are formulated as a frozen solid, (e.g., HOCl ice). The use of frozen HOCl ice may be used in the prevention, alleviation, reduction or treatment of an inflammatory disorder. In such embodiments, the frozen HOCl ice provides all of the anti-inflammatory attributes described above, while also providing the anti-inflammatory abilities of icing tissues that are experiencing inflammation. Significantly, synergistic anti-inflammatory effects are achieved from the use of frozen HOCl ice that produce a combined effect greater than the sum of their separate effects of HOCl administration and icing tissues that are experiencing inflammation.

Notably, in some embodiments where frozen HOCl ice is used in the prevention, alleviation, reduction or treatment of an inflammatory disorder, the frozen HOCl ice makes actual contact with the inflamed tissue, rather being separated from the inflamed tissue plastic barrier or other non-permeable barrier. In other embodiments, a permeable barrier is used between the frozen HOCl ice and the inflamed tissue. In such embodiments, the permeable barrier enables the cold temperature to travel through the permeable barrier without direct contact with the inflamed tissue to prevent tissue damage due to the cold temperature. Additionally, the permeable barrier enables gaseous or liquid HOCl from the sublimation or melting of the frozen HOCl ice.

In some embodiments, the compositions of the present disclosure can be formulated as pharmaceutical, cosmetic or dermatological compositions, and can include a pharmaceutically acceptable carrier. Examples of suitable carriers include, but are not limited to, clays, hectorite, silicates, fluorosilicates, bentonite, oil emulsions, cyclomethicone, polyvinyl alcohol, povidone, hydroxypropyl methyl cellulose, poloxamers, carboxymethyl cellulose, hydroxyethyl cellulose, and purified water. The composition may also include various other ingredients, such as surfactants, co-solvents, viscosity enhancing agents, preservatives, and other therapeutic agents. Examples of viscosity enhancing agents include, but are not limited to, pharmaceutically-acceptable silicates for topical application, polysaccharides, such as hyaluronic acid and its salts, chondroitin sulfate and its salts, dextrans, various polymers of the cellulose family; vinyl polymers; and acrylic acid polymers, etc. For example, the composition may exhibit a viscosity of 1 to 400,000 centipoises (“cps”). In some embodiments, the composition is a hydrogel comprising a silicate-based carrier (e.g., fluorosilicate carrier). For example, the silicate can comprise a fluorosilicate salt such as sodium magnesium fluorosilicate or sodium lithium magnesium fluorosilicate. The hypohalous acid solution can be used as a dispersing media with the silicate carrier to prepare the hydrogel. The formulation may be a hydrogel having a conductivity of from about 0.5 mS/cm to about 12 mS/cm, such as from about 1 mS/cm to about 10 mS/cm in some embodiments. The hydrogels may be prepared from silicate-based carriers, such as 0.5% to about 5% sodium magnesium fluorosilicate.

In some embodiments, the compositions of the present disclosure can be formulated for the aerosol delivery by inhalation of highly aqueous liquid compositions to the respiratory tract.

C. Treatment of Inflammatory Disorder and Cancer with Hypochlorous Acid Compositions

Authentic, pure, hypohalous acid compositions are remarkably benign in their effects on tissues and physiological systems, permitting their use topically on skin and mucous membranes to control infection and enhance healing. Aerosolized hypohalous acid can also be safely inhaled over prolonged periods as a means of delivering active compound to both upper and lower respiratory epithelial surfaces, as well as likely delivery into the systemic circulation as a result of transmembrane mobility of hypohalous acid, or via generation of substrate reaction products such as N-chlorotaurine and chlorinated tyrosine residues. These bioactive entities show antimicrobial properties that are much longer lived than hypohalous acid though much less potent. However, they may also function as pharmacophores at sites remote from the respiratory mucosae, bringing about modifications that mimic those initiated by authentic hypohalous acid during pathological events, particularly those involving inflammation-mediated damage.

Accordingly, exogenous hypohalous acid can be used as an effector to modify powerful biochemical mediators such as cytokines, including IL-6, and prevent their participation in hyper-reactivities that endanger normal tissues and are capable of inflicting serious damage on those physiological processes needed to control infections and restore normal functions and tissue architecture after injury. IL-6 is a polypeptide with affinities for receptor sites on the surface of responsive cells, triggering transmembrane signals that drastically alter cell behaviors, sometimes to the detriment of those cells and the surrounding tissues. Certain binding areas on the cytokine permit complex formation with cell membrane receptors, and upon exposure to hypohalous acid irreversible modifications lead to ablation of the association of IL-6 with these receptors, and the usual subsequent transmembrane signaling. Antigenic regions recognized by IL-6 specific antibodies become modified by hypohalous acid under conditions that are compatible with in vivo efficacy of exogenously supplied hypohalous acid. These changes in antigenicity also occur upon exposure to hypohalous acid, and they prevent the recognition of epitopes by antibody reagents that are IL-6 specific.

By modifying IL-6 and rendering it incapable of interacting with cell surface receptors these authentic pure hypohalous acid compositions demonstrate the potential for preventing IL-6 mediated pathogenesis, thereby offering novel means of neutralizing its adverse biological effects so as to prevent disease or treat disease processes already underway. Reducing the local availability of this cytokine through use of readily manufactured hypohalous acid solutions that beneficially impact tissue defense and repair without causing adverse effects can contribute in important and timely ways to the management of inflammatory conditions as diverse as COVID-19 virus infection cytokine storms, rheumatoid arthritis, or fibromyalgia. Regimens of regular exposure to hypohalous acids may also serve to lower systemic IL-6 levels that contribute to the replication of cytokine-mediated growth of malignancies, including prostatic cancers. They also affect a variety of chemical growth factors that contribute to the replication of carcinomata and sarcomata in many tissues of the body.

Anti IL-6 agents based on the antagonism of antibodies directed against either cytokine RBDs or against the cell membrane receptors that complex with IL-6 are known to provide clinically important benefits. The use of exogenous hypochlorous acid or hypobromous acid compositions to neutralize adverse effects of IL-6 in vivo provides an alternative approach to this antagonism by delivering comparably advantageous changes in a more convenient and economically attractive manner. Other interleukins and chemokines are known to participate in proinflammatory events in vivo, such as IL-2, IL-7, Tumor Necrosis Factor (TNF), and the entire class of chemokine proteins. Their functions in vivo are also likely to be subject to modification and inhibition by mechanisms similar to those demonstrated herein for IL-6. Certain malignancies also show growth dependencies on interleukins other than IL-6 (for example IL-2 dependent leukemias) and those conditions can also be expected to be affected in vivo by exposure to hypohalous acids in the manner proposed for IL-6.

Accordingly, one embodiment of the present disclosure relates to use of the above disclosed composition comprising a hypohalous acid, which is substantially free of hypochlorite and hypobromite for prevention, alleviation, reduction or treatment of an inflammatory disorder.

Another embodiment of the present disclosure relates to use of the above disclosed composition comprising a hypohalous acid, which is substantially free of hypochlorite and hypobromite for prevention, alleviation, reduction or treatment of a cancer.

The disclosed methods comprise administering to a subject (e.g., a human patient or animal) an effective amount of the above disclosed composition comprising a hypohalous acid, which is substantially free of hypochlorite and hypobromite.

In some embodiments, the subject is a human. In other embodiments, the subject is in need of prevention, alleviation, reduction or treatment of inflammatory disorders resulting from a condition derived from rheumatoid arthritis, polymyalgia rheumatica, fibromyalgia, myalgic encephalomyeltis, multiple sclerosis, optic neuritis, acute respiratory distress syndrome (ARDS), disseminated intravascular coagulation, acute smoke inhalation, COVID-19 or combinations thereof. The above disclosed hypohalous acid composition neutralizes the adverse biological activities of IL-6 in vivo. In still other embodiments, the subject is in need of inhibition of the growth of a cancer. In some embodiments, the cancer is prostate cancer, multiple myeloma, plasma cell leukemia, or carcinoma, such as any of those arising from epithelial cells in tissues, including renal, pancreatic and cervical epithelia, and ovarian sarcomata. The above disclosed hypohalous acid composition inactivates IL-6 in vivo responsible for the growth of certain malignancies.

The compositions of the present disclosure can be administered by using various methods. For example, the disclosed compositions can be administered topically or by inhalation.

In some embodiments, the compositions are administered topically to a subject, e.g., by the direct laying on or spreading of the composition on the epidermal or epithelial tissue of the subject. Such topical application can be locally administered to any affected area, using topical administration. Such modes of administration include, but are not limited to, as an ointment, gel, lotion, or cream base or as an emulsion, as a patch, dressing or mask, a nonsticking gauze, a bandage, a swab or a cloth wipe. The composition may be applied to affected areas as needed to combat and/or control disease symptoms, or may be applied using a more precise regimen, such as about daily, or from 1 to about 10 times per day, or from 1 to about 4 times per day, or from 1 to about 3 times per day (e.g., about twice per day).

In some embodiments, the compositions are administered via inhalation by any suitable means known to those of skill in the art for administering therapeutic compositions via inhalation, or via intra-lesional inoculation, or systemically via intravenous or intra-arterial inoculation. The composition may be formulated as an aerosol or mist spray. Such formulations may be produced in a convention manner using appropriate liquid carriers. The composition may be aerosolized with a nasal spray dispenser. The composition can be delivered by various devices known in the art. The aerosol spray composition can be delivered by an intranasal pump dispenser or squeeze bottle. The composition can also be inhaled via a metered dose inhaler. The composition may be administered as needed to combat and/or control disease symptoms, such as about daily, or from 1 to about 10 times per day, or from 1 to about 4 times per day, or from 1 to about 3 times per day (e.g., about twice per day).

The following examples are provided for purposes of illustration, not limitation.

### EXAMPLES

### Example 1

### Preparation of Authentic Pure and Stable Hypochlorous Acid and Hypobromous Acid

A hypochlorous acid (HOCl) composition useful in the methods of the invention, BrioHOCl™, was supplied by Briotech Inc., Woodinville, Wash. Briefly, HOCl results from electrolysis of an aqueous solution of sodium chloride so as to provide at the anode conditions that attract and stabilize reaction products that form HOCl. The end-product is a solution with a range of pH upon packaging and storage of 3.8-4.5 at warehouse environmental temperatures (3.5° C. to 35° C.), an oxidative reduction potential (ORP) ORP of +1100 mV, a salt (NaCl) concentration of either 0.85% to 2% by weight, and preferably 0.85% by weight, and a free chlorine concentration of preferentially 250-300 mg/L at the time of production. No adjustments are ever made to this HOCl solution by the addition of buffers, metal ions, organic heterocyclic halogen stabilizers or pH modifiers of any sort, at any level. Representative methods to prepare authentically pure and stable HOCl is described in U.S. Application No. 63/062,287 and U.S. application Ser. No. 17/396,018. In these applications, an automated and remotely monitored and controlled system is described that receives locally sourced salt and water, processed through an onboard filtration and mixing system, flowing through a water implosion device and a specialized brine electrolysis reactor to automatically produce a consistent pharmaceutical-level, precisely controlled, pH-, ORP- and HOCl-concentration-defined product. This resilient asset in a hardened deployable distributed manufacturing system allows for strategic response of a consistently effective and safe solution to virtually any global locale.

Hypobromous acid (HOBr) was prepared by addition of equimolar quantities of sodium bromide (NaBr) to HOCl, resulting in complete conversion of HOCl to HOBr detectable spectrophotometrically by absorption at 250 nm.

### Example 2

### Inhibition of IL-6 Reactivity with Specific Antibodies by Exposure to Authentic Pure HOCl or HOBr

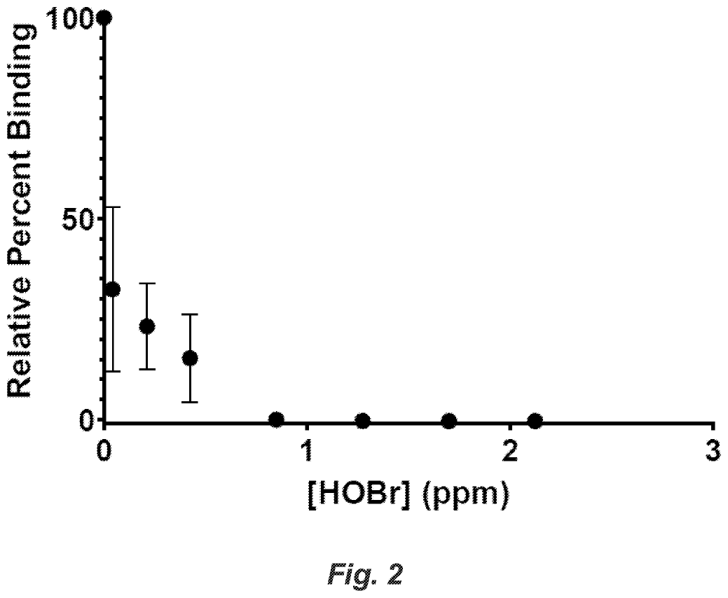

An ELISA assay (Fisher Scientific) was used to detect binding changes between IL6 and anti-IL6 antibody binding in the presence of HOCl or HOBr. Anti-human IL6 antibody was immobilized to a polystyrene 96 well plate as the capture antibody. IL-6 was exposed to various concentrations of HOCl or HOBr for 5 minutes. The active halogen was quenched before incubation with the coated plate wells. A second anti-human IL6 antibody was introduced to the coated wells and served as a detector to an enzyme-mediated chromophore indicator system. At HOCl concentrations >2 ppm there was no detectable binding to the capture antibody. At >2 ppm in the presence of HOBr, binding was also undetectable, as shown in FIG. 2.

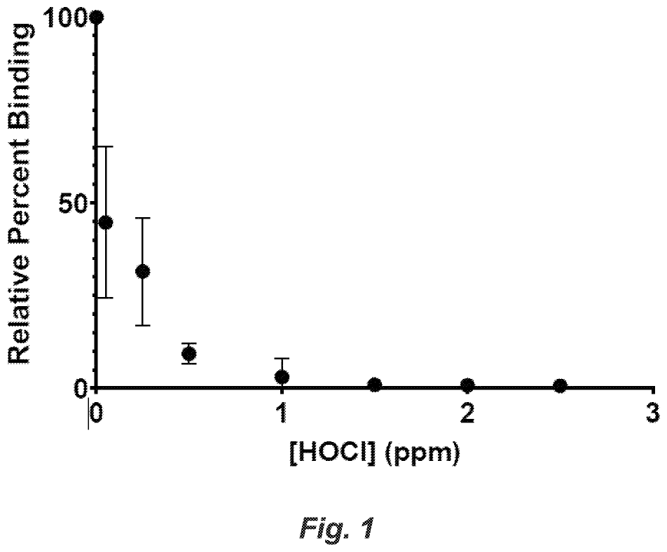

The inhibition of IL-6 reactivity by exposure to HOCl is illustrated in FIG. 1. The results demonstrate that exposure of IL-6 to HOCl or HOBr solutions results in rapid modification of epitopes on the cytokine recognized by specific antibodies.

### Example 3

### Inhibition of Cell Receptor Binding by IL-6 after Exposure to Pure HOCl

An IL-6 bioassay (Promega) was used to determine the effect of HOCl on IL-6 binding to human cells triggering a change in luminescence. IL-6 was incubated with various concentrations of HOCl and quenched, before addition to the receptor-expressing human cells. Binding of the cytokine was measured by luminescence. At all concentrations tested, HOCl-treated IL-6 exhibited significantly reduced luminescence. The results demonstrate that exposure of IL-6 to HOCl results in complete inhibition of the capacity of the cytokine to initiate the transmembrane signaling necessary for triggering intracellular responses.

### Example 4

### Safety of Pure HOCl upon Exposure of Intact Skin and Mucous Membranes, and after Exposure Orally or by Inhalation in Experimental Animals

Experimental exposures of rodents were done according to OECD method 423 for acute oral toxicity assessment, OECD method 434 for acute dermal toxicity and OECD method 433 for acute inhalation. Results are shown in Table 1.

TABLE 1
Results of HOCl Safety Testing

| | | | | Result/ |
|---|---|---|---|---|
| | | | Result/ | Histo- |
| System | Organism | Duration | Behavior | pathology |
| Dermal | Mammal | 14 days exposure | No change | No |
| | (mouse) | | | pathology |
| Respiratory | Mammal | 4 hours exposure, | No change | No |
| | (mouse) | evaluated at 14 days | | pathology |
| Gastro- | Mammal | one time exposure | No change | No |
| intestinal | (rat) | to 5000 mg/kg of | | pathology |
| | | aqueous product, | | |
| | | evaluated at 14 days | | |

The results show that exposure of animals to pure stable HOCl induces no detectable pathological changes by any of the tested routes.

### Example 5

### Safety of Human Subjects upon Exposure to HOCl via Inhalation

The safety of exposure of human subjects to microaerosolized pure stable HOCl via the respiratory route was evaluated.

Human volunteers were exposed to dense microaerosols of HOCl for periods of 2-5 minutes and submitted subjective reports of the outcomes of these experiences. Data on a total of 400 such episodes were collected under medical supervision. No serious adverse effects were recorded, and minor complaints (nose irritation, slight impact on ease of deep breathing) were limited to approximately 3%, which disappeared upon cessation of exposure to HOCl.

The results support the safety of human subject exposure for brief periods to pure HOCl by the respiratory route.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for one or more of preventing, alleviating, reducing, and treating an inflammatory disorder, the method comprising administering to a subject an effective amount of a composition comprising a hypohalous acid, wherein the composition does not contain a detectable amount of hypochlorite and hypobromite as measured by Raman spectroscopy, wherein the composition inhibits biological activities of Interleukin 6 (IL-6) and other proinflammatory Interleukins and chemokines in vivo.

2. The method of claim 1, wherein the inflammatory disorder is derived from one or more of rheumatoid arthritis, polymyalgia rheumatica, fibromyalgia, myalgic encephalomyeltis, multiple sclerosis, optic neuritis, acute respiratory distress syndrome (ARDS), disseminated intravascular coagulation, acute smoke inhalation, and COVID-19.

3. A method for one or more of inhibiting and reducing the growth of a cancer, the method comprising administering to a subject an effective amount of a composition comprising an hypohalous acid, wherein the composition does not contain a detectable amount of hypochlorite and hypobromite as measured by Raman spectroscopy, and wherein the composition is administered one or more of systemically, topically, and intra-lesionally.

4. The method of claim 3, wherein the cancer is selected from a group that includes prostate cancer, multiple myeloma, plasma cell leukemia, ovarian sarcomata, renal, pancreatic, cervical carcinomata, and carcinomata arising from other tissue epithelia.

5. The method of claim 3, wherein the composition is an aqueous solution having a hypohalous acid concentration from about 5 mg/L to about 5000 mg/L.

6. The method of claim 3, wherein the composition is an aqueous solution of HOCl having a pH from about 3.8 to about 6.5.

7. The method of claim 6, wherein the composition has a pH from about 4.0 to about 4.2.

8. The method of claim 3, wherein the composition is an aqueous solution having an oxidative reduction potential (ORP) from about +900 milivolts to +1200 milivolts.

9. The method of claim 3, wherein the hypohalous acid is hypochlorous acid.

10. The method of claim 9, wherein the composition has a hypochlorous acid concentration from about 5 mV to about 500 mg/L, a pH from about 3.8 to about 6.5 and an ORP of about +1000 milivolts.

11. The method of claim 9, wherein the composition has a hypochlorous acid concentration from about 80 mg/L to about 300 mg/L, a pH from about 4.0 to about 4.2, and an ORP of about +1100 milivolts.

12. The method of claim 9, wherein the composition has a hypochlorous acid concentration from about 80 mg/L to about 300 mg/L, a pH from about 4.0 to about 4.2, and an ORP of about +1138 mV.

13. The method of claim 3, wherein the hypohalous acid is hypobromous acid.

14. The method of claim 13, wherein composition has a hypobromous acid concentration from about 10 mg/L to about 300 mg/L, a pH from about 4.0 to about 7.5, an oxidative reduction potential (ORP) of about +1000 millivolts.

15. The method of claim 13, wherein composition has a hypobromous acid concentration from about 5 mg/L to about 350 mg/L, a pH from about 4.0 to about 7.5, an oxidative reduction potential (ORP) of about +900 millivolts.

16. The method of claim 3, wherein the composition is formulated for one or more of topical administration and administration via inhalation.

17. The method of claim 3, wherein the composition is formulated as a liquid, a gel, a lotion, a cream, a foam, a gas, or an aerosol spray.

18. The method of claim 17, wherein the composition further comprises a pharmaceutically acceptable carrier.

19. The method of claim 18, wherein the carrier comprises a clay, a cellulose, a silicate or a combination thereof.

20. The method of claim 3, wherein the composition is administered from 1 to 10 times per day.

21. The method of claim 20, wherein the composition is administered from 1 to 4 times per day.

22. The method of claim 3, wherein the composition is an aqueous hypochlorous acid composition prepared by electrolysis of an aqueous solution of sodium or potassium chloride.

23. The method of claim 22, further comprising converting the hypochlorous acid to hypobromous acid prior to the administering the composition to the subject.

24. The method of claim 23, wherein converting the hypochlorous acid to the hypobromous acid comprises mixing the hypochlorous acid composition with an aqueous solution of sodium or potassium bromide in equimolar amounts.

25. The method of claim 1, wherein the composition is an aqueous solution having a hypohalous acid concentration from about 5 mg/L to about 5000 mg/L.

26. The method of claim 1, wherein the composition is an aqueous solution of HOCl having a pH from about 3.8 to about 6.5.

27. The method of claim 1, wherein the composition is formulated for one or more of topical administration and administration via inhalation.

28. The method of claim 1, wherein the composition is formulated as a liquid, a gel, a lotion, a cream, a foam, a gas, a frozen solid, or an aerosol spray.

29. The method of claim 1, wherein the administration of the compound reduces local availability of cytokine to prevent, alleviate, reduce, or treat the inflammatory disorder, wherein the inflammatory disorder is a COVID-19 virus infection cytokine storm.
